(19) United States
(12) Patent Application Publication
(10) Pub. No.: US 2006/0257374 A1
(43) Pub. Date: Nov. 16, 2006

(54) Use of bdellovibrionaceae as an antimicrobial agent

(76) Inventors: Henry Neal Williams, Tallahassee, FL (US); Silvia Pineiro, Columbia, FL (US)

Correspondence Address: FLORIDA A&M UNIVERSITY, 2077 E. PAUL DIRAC DRIVE, TALLAHASSEE, FL, 32310, US

(21) Appl. No.: 11/127,636

(22) Filed: May 12, 2005

(51) Int. Cl. A61K 35/74; A61K 39/02

(52) U.S. Cl. 424/93.4; 424/234.1

(57) ABSTRACT

Compositions and methods are provided for the treatment of bacterial infections with live bacterial agents. The invention also provides methods of producing bacterial preparations for medicinal use. The inventive compositions and methods can be useful in preventing or treating bacterial infections in multicellular organisms by infecting and lysing the bacterial target.

This invention was supported with funding from the National Science Foundation (Grant No. 0455276). The United States Government has certain rights in the invention.

### FIELD OF THE INVENTION

The present invention relates generally to antibiotic medicaments and treatments. More particularly, the present invention relates to compositions and methods of using live bacteria as antibiotic therapeutic agents.

### BACKGROUND OF THE INVENTION

Bacterial infections can afflict multicellular organisms, such as, for example, humans at any stage of development. Among children, the elderly, and otherwise immunocompromised individuals, however, bacterial infections can be especially dangerous. Ear infections, by way of example, are relatively common, especially in children and are the leading cause of visits by children to physicians. Particularly where an individual suffers from chronic bacterial infections and are given conventional antibacterial medicaments, he or she is at greater risk at developing bacteria that are resistant to the conventional medicaments. In fact, bacteria that are prone to becoming resistant to antibiotics are known to cause ear infections, like many other infections. Therefore, there is a need for novel medicaments and approaches to treating bacterial infections.

### SUMMARY OF THE INVENTION

The foregoing needs are met, to an extent, by the present invention, wherein in one aspect a method is provided of treating or preventing a bacterial infection in a subject comprising administering a therapeutically effective amount of one or more strains of Bdellovibrionaceae (Bd) to the subject. The one or more strains of Bd may be Bdellovibrio bacteriovorus, Bacteriovorax stolpii, Bacteriovorax starrii, Bacteriovorax marinus, and/or Bacteriovorax litoralis. The bacterial infection may comprise a gram negative bacterium and in a mammal, optionally a human. Bacterial infections include ear infections, ear infections within the ear canal. In the case of such ear infections, the administration can be via droplets into the ear canal. Other bacterial infections include, for example, skin infections, burn infections, eye infections, and gastrointestinal infections.

In another embodiment of the present invention, a pharmaceutical composition is provided comprising a therapeutically effective amount of one or more strains of Bdellovibrionaceae (Bd) and a pharmaceutically acceptable carrier. The one or more strains of Bd may be Bdellovibrio bacteriovorus, Bacteriovorax stolpii, Bacteriovorax starrii, Bacteriovorax marinus, and/or Bacteriovorax litoralis. Pharmaceutically acceptable carriers may include sterile seawater, sterile physiological saline, or sterile distilled water.

In still another embodiment of the present invention, a pharmaceutical composition is provided comprising a therapeutically effective amount of one or more strains of lyophilized Bdellovibrionaceae (Bd).

In further still another embodiment of the present invention, a method of preparing a pharmaceutical composition is provided comprising one or more strains of Bdellovibrionaceae (Bd) comprising: inoculating a culture of starter bacteria, capable of being infected by a strain of Bd, with at least one strain of Bd to provide a source of Bd; removing from the culture substantially all of the starter bacteria; and suspending the Bd cells substantially free of the starter bacteria in a solution at a predetermined concentration. The strain of Bd may be selected from the group consisting of Bdellovibrio bacteriovorus, Bacteriovorax stolpii, Bacteriovorax starrii, Bacteriovorax marinus, and/or Bacteriovorax litoralis. The starter bacteria can be gram negative and/or attenuated. In some embodiments, the method may include the removal of substantially all of the starter bacteria accomplished in a centrifuge. The solution may be sterile seawater, sterile physiological saline, or sterile distilled water. The predetermined concentration may be about 10⁹ to about 10¹² cells per mL, and preferably about 10¹⁰ cells per mL. The method may further comprise freezing the solution of Bd cells substantially free of the starter bacteria or lyophilizing the solution of Bd cells substantially free of the starter bacteria.

There has thus been outlined, rather broadly, certain embodiments of the invention in order that the detailed description thereof herein may be better understood, and in order that the present contribution to the art may be better appreciated. There are, of course, additional embodiments of the invention that may be described below and which may form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art may appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is table depicting the susceptibility of clinical bacterial isolates to freshwater Bd.

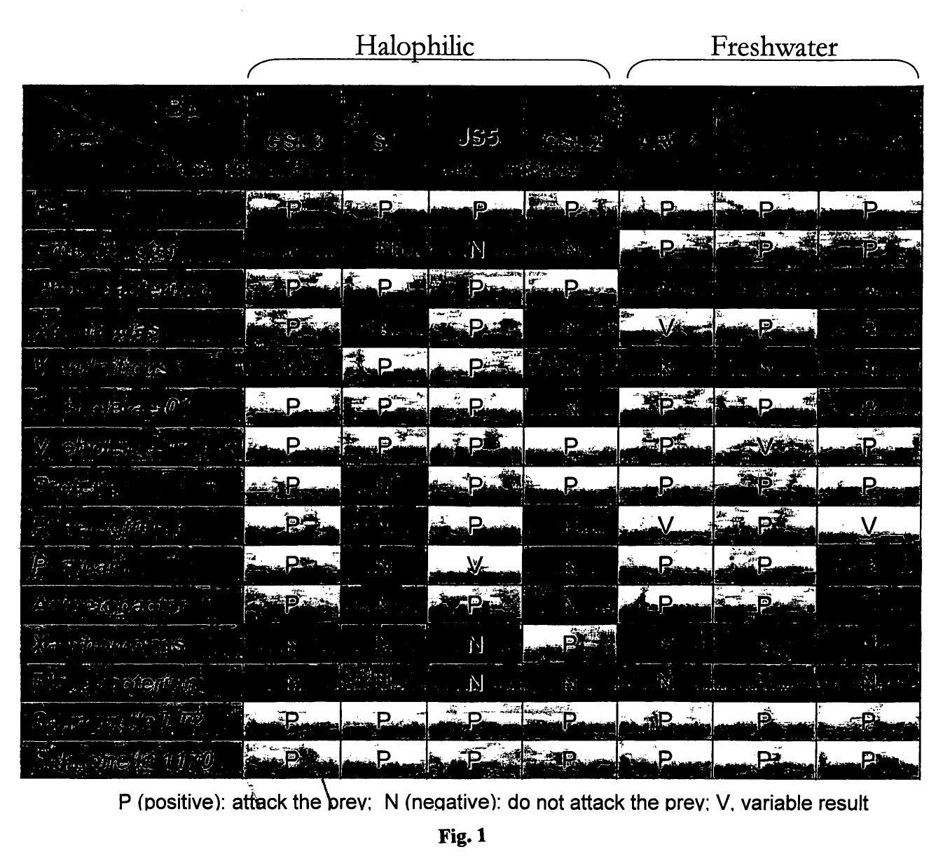

FIG. 2 is table depicting the susceptibility of clinical bacterial isolates to HBd.

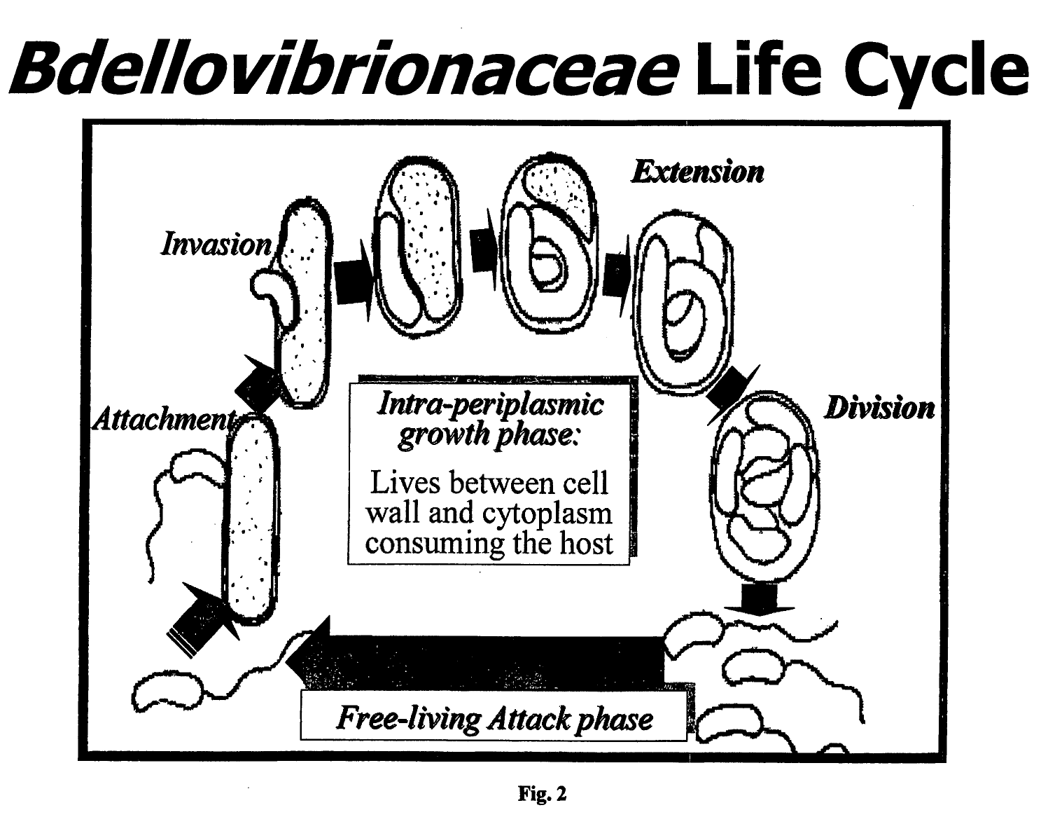

### DETAILED DESCRIPTION

The present invention relates to the use of strains of Bdellovibrionaceae (Bd) for treating bacterial infections. Briefly, Bd are Gram negative, prokaryotic bacteria, about 1-about 2 μm in length, and about 0.25-about 0.4 μm in width. Bd have a relatively small genome estimated at about 3.8 Mb, have a biphasic life cycle, and exist in both marine and freshwater forms.

While it is currently thought that Bd is ineffective in treating Gram positive bacteria, many strains of Bd have yet to be identified or are still poorly understood. Accordingly, the scope of the present invention should be understood to include the use of Bd strains in treating both Gram negative and positive bacteria. Strains of Bd currently known include: Bdellovibrio bacteriovorus, Bacteriovorax stolpii, Bacteriovoraxstarrii, Bacteriovorax marinus, and Bacteriovorax litoralis. Furthermore, in some embodiments, the novel antibiotics of the present invention may be used in conjunction with conventional antibiotics known in the art.

Compositions and methods of treatment in accordance with the present invention may include the use of one or more strains of Bd alone or in combination. In selecting the optimal Bd strain, it may be necessary to test multiple Bd isolates from the same environment. For example, in one study, of three isolates from soil only one was effective in reducing populations of P. glycinea on soybean leaves. In other instances, it may be necessary to test multiple Bd isolates from different environments. For example, it has been shown that Salt Lake Bd isolates have different prey susceptibility patterns than ocean or estuarine isolates. Additional factors to consider may be whether the target bacteria causing infection are susceptible to the Bd strain, whether the Bd strain can kill target bacteria but substantially not beneficial ones, the Bd load required to be effective, and whether the Bd strain is active in the environment to be treated (e.g., temperature, salinity).

One method of producing Bd suspensions will now be described. Bd may be grown in broth culture using an attenuated strain of bacteria as “prey,” or “starter” bacteria. An attenuated strain is preferred in some embodiments as one of precautionary steps to reduce the possibility of the prey causing infection should it be accidentally introduced to the subject with Bd. However, the prey may be removed from the final suspension and should not be introduced into the ear as will be described.

Growth of Bd prey or “starter” microorganisms. V. parahaemolyticus P-5 has been used as the prey or “starter” culture for the routine cultivation of halophilic bdellovibrios. P-5 is grown on Sea Water Yeast Extract Agar (SWYE) (T. Kaneko and R. R. Colwell, 1978, Microbial Ecology 4:135-155) at 25° C. For growth of the terrestrial or freshwater Bd E. coli ML 35 is used. E. coli is grown on Trypticase Soy Agar (Difco) at 25° C. In both cases a heavy inoculum picked from a colony of the organisms from a previous culture plate is inculated onto a fresh culture plate using the streak plate method to yield nearly confluent growth on most areas of the plate.

After 24 h incubation, the P-5 “starter” bacteria were harvested from the surface of the agar plate by flooding with 9 ml of sterile 70% artificial seawater (Instant Ocean, Aquarium Systems, Mentor, Ohio 44060) and suspending growth of bacteria on the plate into the liquid using sterile glass “hockey sticks”. The E. coli or other terrestrial “starter” bacteria prey are harvested in the same manner, replacing the ASW with sterile distilled water. The suspensions are then adjusted to contain approximately 10⁹ organisms/ml (O.D. of 0.85 at 600 nm). The starter bacterium is the sole source of nutrients for the Bd. Other “starter” bacteria may be used include other Vibrio species such as V. vulnificus, V. anquillarum, and Photobacterium species for the halophilic Bd. Other “Starter cultures” that are used for growing the terrestrial/freshwater Bd include species of Proteus, Acinetobacter, Salmonella and other E. coli strains.

Bd is then introduced to the starter culture. The Bd source may be from a previously grown or frozen stock culture which can be used to inoculate a turbid suspension of prey cells, approximately 5×10⁸ cells per mL. The culture may be incubated on a rotary shaker at between 23 to 25° C. until the turbid suspension has become clear which is indicative of lyses of the prey by the Bd. The suspension may be examined microscopically to confirm the killing and lysis of the prey cells and the presence of Bd at a desirable concentration. The suspension may then be centrifuged at low speed to sediment the remaining prey cells and larger pieces of debris.

Following centrifugation the supernatant fluid may be filtered through 0.2 μm pore size filter to remove most remaining prey and debris. The filtrate may again be centrifuged at high speed to pellet or concentrate the Bd cells. Following this centrifugation, the supernatant fluid may be discarded. The remaining pellet may be resuspended in a small volume of sterile diluent. Optionally, the suspensions may be centrifuged as before and the process repeated, for example, three times. This repetition is done to “wash” the Bd cells substantially free of any contaminant material and to yield a final suspension of preferably approximately 5 to 10 mL.

The Bd may be counted by any known method in the art, including fluorescent direct microscopy, to determine the number of cells present. The suspension may then be either further concentrated or diluted as the case may be to yield a preferably standard preparation of 10¹⁰ cells per mL in either sterile seawater, physiological saline or distilled water, depending upon the requirements of the Bd genera, species or strain used.

The suspension can be shipped to a physician's clinic in one of several forms, including refrigerated, frozen, or freeze-dried. The refrigerated suspension should be kept at 5 to 7° C. for optimal shelf life, which may extend to three or more weeks. Frozen suspensions may be delivered to the physician's clinic without thawing and preferably be maintained in that state until a few minutes prior to administration. The shelf life for the frozen preparation may be six or months. Freeze-dried preparations may have the longest shelf life, of approximately one or more years. Prior to use, the physician should reconstitute this preparation by adding about 5 mL of one of the diluents described above depending on the genus, species or strain of Bd. Once reconstituted, the Bd preparation may be administered as the suspensions above and may thereafter be maintained under refrigeration until the treatment is complete.

When treating ear infection, in some embodiments, the frozen suspensions may be thawed and administered by placing approximately one to ten, and preferably about three drops in the patient's ear. The patient should keep the head tilted for about five minutes to prevent the suspension from running out of the ear canal. Any remaining suspension can be given to the patient with instructions to keep refrigerated and administer the drops as required. In some cases, this requirement may be about five times daily for about four days.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

What is claimed:

1. A method of treating or preventing a bacterial infection in a subject comprising administering a therapeutically effective amount of one or more strains of Bdellovibrionaceae (Bd) to said subject.

2. The method of claim 1 wherein the one or more strains of Bd is selected from the group consisting of Bdellovibrio bacteriovorus, Bacteriovorax stolpii, Bacteriovorax starrii, Bacteriovorax marinus, and Bacteriovorax litoralis.

3. The method of claim 1 wherein the bacterial infection comprises a gram negative bacterium.

4. The method of claim 1 wherein the subject is a mammal.

5. The method of claim 4 wherein the mammal is a human.

6. The method of claim 1 wherein the bacterial infection is an infection selected from the group consisting of an ear infection, skin infection, burn infection, eye infection and gastrointestinal infection.

7. The method of claim 1 wherein the ear infection is with an ear canal.

8. The method of claim 7 wherein the administration is via droplets into the ear canal.

9. A pharmaceutical composition comprising a therapeutically effective amount of one or more strains of Bdellovibrionaceae (Bd) and a pharmaceutically acceptable carrier.

10. The pharmaceutical composition of claim 13 wherein the one or more strains of Bd is selected from the group consisting of Bdellovibrio bacteriovorus, Bacteriovorax stolpii, Bacteriovorax starrii, Bacteriovorax marinus, and Bacteriovorax litoralis.

11. The pharmaceutical composition of claim 13 wherein the pharmaceutically acceptable carrier is sterile seawater, sterile physiological saline, or sterile distilled water.

12. A pharmaceutical composition comprising a therapeutically effective amount of one or more strains of lyophilized Bdellovibrionaceae (Bd).

13. A method of preparing a pharmaceutical composition comprising one or more strains of Bdellovibrionaceae (Bd) comprising:
inoculating a culture of starter bacteria, capable of being infected by a strain of Bd, with at least one strain of Bd to provide a source of Bd;
removing from the culture substantially all of the starter bacteria; and
suspending the Bd cells substantially free of the starter bacteria in a solution at a predetermined concentration.

14. The method of claim 17 wherein the strain of Bd is selected from the group consisting of Bdellovibrio bacteriovorus, Bacteriovorax stolpii, Bacteriovorax starrii, Bacteriovorax marinus, and Bacteriovorax litoralis.

15. The method of claim 17 wherein the starter bacteria is gram negative.

16. The method of claim 17 wherein the starter bacteria is attenuated.

17. The method of claim 17 wherein the removal of substantially all of the starter bacteria is accomplished in a centrifuge.

18. The method of claim 17 wherein the solution is sterile seawater, sterile physiological saline, or sterile distilled water.

19. The method of claim 17 wherein the predetermined concentration is about 10⁹ to about 10¹² cells per mL.

20. The method of claim 23 wherein the predetermined concentration is about 10¹⁰ cells per mL.

21. The method of claim 17 further comprising freezing the solution of Bd cells substantially free of the starter bacteria.

22. The method of claim 17 further comprising lyophilizing the solution of Bd cells substantially free of the starter bacteria.
